(19) United States
(12) Patent Application Publication
(10) Pub. No.: US 2004/0101939 A1
(43) Pub. Date: May 27, 2004

(54) Method for reducing or preventing modification of a polypeptide in solution

(76) Inventors: Ling C. Santora, Shrewsbury, MA (US); Krista H. Stanley, Fiskdale, MA (US)

Correspondence Address: ABBOTT BIORESEARCH, 100 RESEARCH DRIVE, WORCESTER, MA, 01605-4314, US

(21) Appl. No.: 10/302,592

(22) Filed: Nov. 22, 2002

(51) Int. Cl. C07K 16/24; C12N 9/00; C07K 1/06

(52) U.S. Cl. 435/183; 530/412; 530/388.23

(57) ABSTRACT

The present invention encompasses a novel approach to reduce or to prevent modification of polypeptides in solution and to polypeptides obtained by such methods. Specifically, the invention relates to a method for reducing or preventing modification of polypeptides in milk, particularly milk obtained from a transgenic animal, and to polypeptides isolated using such methods.

### FIELD OF THE INVENTION

The present invention relates to methods for reducing or preventing polypeptide modification in solution. Specifically, the invention relates to a method for reducing or preventing modification of polypeptides in milk, particularly milk obtained from a transgenic animal.

### BACKGROUND OF THE INVENTION

The biotechnology industry is faced with the challenge of producing purified proteins on a large scale. Recombinant technology has made it possible to express proteins in various biological systems such as bacteria, yeast, and animal cell culture. An alternate system is animal-based production, wherein transgenic animals produce and express a desired protein into a body fluid (e.g., serum, milk, and urine). Mammals, particularly dairy animals, are especially suited for large-scale production of protein in milk. (Pollock, et al., 1999).

Recombinant proteins have been successfully produced in milk of transgenic animals. (Houdebine et al., 2000). Recombinant proteins from milk are often produced as whey proteins. Using standard centrifugation, membrane filtration and chromatographic procedures the recombinant proteins are isolated from milk.

Recombinant proteins expressed in cell culture and transgenic animal production systems are heterogeneous, however. This heterogeneity is often seen in proteins expressed in milk. Milk proteins and their modifications in milk, skim milk, and infant formulae have been characterized using ESI/MS and MALDI/MS coupled with HPLC or CE (Siciliano et al., 2000; Sabbadin et al., 1999; Galvani et al., 2000; Catinella et al., 1996a & 1996b; Jones et al., 1998; and Traldi, 1999). For example, three post-translational modifications of proteins isolated from cow milk have been identified: they include, multiple phosphorylations (+80×Da on Ser or Try), pyrrolidone carboxylic acid modification of Gin, and single/multiple lactosylation (+162 Da on Ser) (Sabbadin et al., 1999).

In addition to post-translational modifications, post-secretional and other modifications may occur when milk is stored for a period of time. Production protocols require large quantities of milk. Collected milk is typically stored, refrigerated, until sufficient quantities are collected for purification. Despite refrigeration, recombinant proteins are often not stable in the milk. Proteins may undergo chemical modification during storage. As disclosed herein, one such modification of polypeptides in milk includes acidic modification.

There is, therefore, a need in the art for efficient and effective methods for reducing or preventing modification of proteins in solution. This invention describes a new purification process developed to reduce or to prevent modification of recombinant proteins in solution, particularly post-secretional modifications of recombinant proteins expressed in transgenic animals. Specifically, the invention provides a method for acidifying a recombinant polypeptide-containing solution prior to, or after, storage, and before isolation of the polypeptide to reduce or to prevent modification of the protein in solution. The use of acid to reduce or to prevent modification (as opposed to separation) of polypeptides in solution, and in particularly in milk, has not been previously described.

### SUMMARY OF THE INVENTION

This invention is directed to a method for identifying modification of polypeptides in solution and particularly a method to reduce or to prevent modification of polypeptides in solution. One embodiment pertains to a method for reducing or preventing modification of polypeptides in a solution comprising the steps of: a) providing a solution containing a polypeptide susceptible to modification; b) adding acid to the solution. Optionally, the solution may be stored at a temperature below room temperature before and/or after the acid-adding step b), and optionally further comprises a final step comprising isolating the polypeptide from the solution.

Preferably the polypeptide-containing solution is a solution obtained from or comprising a bodily fluid of an animal. More preferably the bodily fluid is selected from the group consisting of serum, milk, and urine. Most preferably the solution is milk.

One preferred embodiment of the present invention is directed to a method for reducing or preventing modification of a polypeptide in milk. Preferably, the method comprises the steps of: providing milk containing a polypeptide; adding acid to the milk; optionally storing the milk at a temperature below room temperature (before and/or after the addition of acid); and, preferably, isolating the polypeptide from the milk.

In a further embodiment, milk is obtained from a transgenic animal. Preferably the transgenic animal is a dairy mammal. Alternatively, the transgenic animal is selected from the group consisting of a cow, goat, sheep, pig, rat, and mouse. Most preferably the transgenic mammal is a goat.

The invention pertains to any polypeptide in solution. More preferably, the polypeptide is an antibody. Most preferably the antibody is an anti-TNF antibody, such as D2E7 (as disclosed and taught in Salfeld et al., 2000, and 2001).

Preferably, the amount of acid added to the polypeptide-containing solution (e.g., milk) is sufficient to obtain a pH of about pH 7.0 to about pH 1.0 More preferably, the amount of acid added is sufficient to obtain a pH of about pH 5.0 to about pH 2.0. More preferably, the amount of acid added is sufficient to obtain a pH of about pH 4.0 to about pH 3.0. Ideally, sufficient acid is added such that the pH of the polypeptide-containing solution is about pH 3.5 to about pH 3.0.

In a specific embodiment, the acid added to the polypeptide-containing solution is elected from the group consisting of: acetic acid, citric acid, formic acid, and hydrochloric acid. More preferably, the acid is citric acid. Most preferably the acid is 2.5M citric acid.

In a further embodiment of the present invention, the polypeptide-containing solution may be stored for a period of time before and/or after addition of the acid. Preferably, the temperature at which the solution is stored is about 4° C. to about −80° C. Preferably, the temperature is about 0° C. to about −70° C. More preferably, the temperature is about −10° C. to about −50° C. Most preferably, the temperature is about −15° C. to about −30° C. In the embodiment wherein the polypeptide-containing solution is milk, ideally, the temperature at which the milk is stored is about −20° C.

In a most preferred embodiment, the invention provides a method for reducing or preventing modification of D2E7 in milk obtained from a transgenic goat, comprising the steps of: providing transgenic goat milk containing D2E7, and adding 2.5 M citric acid to said milk in an amount sufficient to obtain a pH of said milk of about pH 3.0 to about pH 3.5. Specific embodiments further comprise the optional steps of storing said milk at a temperature below room temperature, and/or isolating D2E7 from the milk.

In a related aspect, the invention provides a polypeptide isolated according to the foregoing method. More specifically the invention is directed to an antibody isolated from milk. The most preferred embodiment is directed to D2E7 isolated from milk obtained from a transgenic animal (e.g., goat).

### BRIEF DESCRIPTION OF DRAWINGS

FIG. 1: represents a chromatographic comparison of CHO D2E7 antibody isoforms with transgenic G-D2E7 antibody isoforms using cation-exchange liquid chromatography (CIEX). Chromatogram B identifies CHO D2E7 peaks (right to left) to be C-terminal 2-Lys, 1-Lys, O-Lys, labeled as 2-K, 1-K and 0-K respectively. Chromatogram A illustrates the peaks in transgenic GD2E7 antibody. The encircled area indicates acidic peaks.

FIG. 2: represents a chromatographic analysis of G-D2E7 and TNFα binding by CIEX. Chromatogram A illustrates G-D2E7 alone. Chromatogram B illustrates TNFα alone. Chromatogram C illustrates a mixture of G-D2E7 and TNFα in excess, and shows formation D2E7TNFα complexes in solution. Chromatogram D illustrates a mixture of G-D2E7 and TNFα, where G-D2E7 is in excess.

FIG. 3: represents a chromatographic comparison of G-D2E7 acidic peaks before and after formic acid treatment by CIEX. Chromatogram A illustrates G-D2E7 without formic acid treatment, containing 42% acidic peaks eluting at 10 minutes. Chromatogram B illustrates G-D2E7 after formic acid treatment.

FIG. 4: represents a chromatographic comparison of G-D2E7, thawed at 4° C., pH 6.5 to 7.0, for 65 hours (A) and 96 hours (B).

FIG. 5: illustrates the effect of temperature and pH on G-D2E7 milk. Line A represents the percentage of G-D2E7 acidic peaks purified from untreated milk at 37° C. for 0, 24, 48, 72 and 96 hour time points. Line B represents the percentage of G-D2E7 acidic peaks purified from untreated milk at room temperature for 0, 24, 48, 72 and 96 hour time points. Line C represents the percentage of G-D2E7 acidic peaks purified from the acid treated milk at room temperature for 0, 24, 48, 72 and 96 hour time points.

FIG. 6: represents a chromatograph of polypeptides isolated after large scale acid precipitation. Chromatogram A illustrates G-D2E7 purified without acid treatment. Chromatogram B illustrates G-D2E7 purified with acid treatment.

### DETAILED DESCRIPTION OF THE INVENTION

The present invention provides a method for reducing or preventing modification of polypeptides in solution. In a preferred embodiment, acid is added to a polypeptide-containing solution to reduce or to prevent modification of the polypeptide. The polypeptide-containing solution may be stored below room temperature before and/or after acid treatment. The polypeptide with reduced or no modification may then be isolated from the acidified solution. In a related aspect, the invention provides a polypeptide isolated according to the method for reducing or preventing modification of polypeptides described herein.

That the present invention may be more readily understood, select terms are defined below.

“Transgenic animal”, as known in the art and as used herein, refers to an animal having cells that contain a transgene, wherein the transgene introduced into the animal (or an ancestor of the animal) expresses a polypeptide not naturally expressed in the animal. A “transgene” is a DNA construct, which is stably and operably integrated into the genome of a cell from which a transgenic animal develops, directing the expression of an encoded gene product in one or more cell types or tissues of the transgenic animal.

“Bodily fluid” as used herein, refers to any fluid obtained from or excreted by an animal. Bodily fluids include but are not limited to; blood, serum, plasma, urine, milk, saliva, nasal secretions, cerebrospinal fluid, lymph fluid, ascites, pleural effusion, fluid obtained from tissue extracts, and intracellular fluid.

“Polypeptide” as used herein, refers to any polymeric chain of amino acids. The terms “peptide” and “protein” are used interchangeably with the term polypeptide and also refer to a polymeric chain of amino acids. The phrase “modification of polypeptide”, as used herein, refers to any addition of one or more radical groups to the polypeptide sequence. For example, polypeptides may be modified by the addition of one or more radical groups such as glycosyl, glucuronidyl, peptidyl, phosphoryl, sulphuryl, farnesyl, acyl, or maleuryl groups.

“Antibody”, as used herein, broadly refers to an immunoglobulin (Ig) molecule comprised of four polypeptide chains, two heavy (H) chains and two light (L) chains or any functional fragment or derivation thereof, which retains the essential epitope binding features of the Ig molecule.

“Post translational modification” refers to any modification or change occurring or existing in a polypeptide after genetic translation of the polypeptide in a cell. “Post secretional modification” refers to any modification or changes occurring or existing in a polypeptide after secretion of polypeptide from a cell into the extracellular environment (such as, but not limited to, bodily fluids and cell culture medium).

“Preventing or reducing” modification of a polypeptide refers to any process which hinders, stops, eliminates, modification of a polypeptide either before such modification occurs or by reversing (e.g., removing) such modification to the polypeptide. Preventing or reducing modification of a polypeptide according to the present invention is a comparative measure of the amount of modification present on the polypeptide relative to the amount of modification present on the polypeptide absent the acid treatment of the present invention. Preferably, the amount of modification of a polypeptide of interest is significantly reduced (i.e., by at least about 5%). More preferably, the amount of modification of a polypeptide of interest is reduced by at least about 10%; more preferably, by at least about 20%; more preferably, by at least about 25%; more preferably, by at least about 50%; more preferably, by at least about 75%; more preferably, by at least about 80%; more preferably, by at least about 90%; more preferably, by at least about 95%. In the most preferred embodiment, preventing or reducing modification of polypeptide is achieved in that the polypeptide of interest (upon treatment as described herein) possesses no post-secretional modification.

The term “acid”, as used herein, includes weak and strong acids capable of reducing the pH of a solution. Examples of such acids include, but are not limited to, acetic acid, citric acid, formic acid, or hydrochloric acid. The phrase “below room temperature”, as used herein, refers to any temperature below about 28° C. Preferable temperatures below room temperature include a range of about 28° C. to about 80° C., more preferably a range of about 15° C. to about −80° C., more preferably a range of about 4° C. to about −20° C., most preferably about −20° C.

I. Expression of Polypeptides in Transgenic Animals

Recombinant polypeptides can be expressed in, for example, microorganisms, plant cells, and animal cells, including transgenic animals. Conventional methods involve inserting the gene responsible for the production of a particular polypeptide into host cells such as bacteria, yeast, or mammalian cells, and growing the cells in culture media. The cultured cells then synthesize the desired polypeptide. Alternatively, transgenic animals can be produced by introducing into developing embryos a transgene, (i.e., a nucleic acid that encodes a polypeptide of interest) such that the nucleic acid is stably incorporated in the genome of germ line cells of the mature animal and inheritable. At least some cells of such transgenic animals are capable of expressing the polypeptide of interest.

Standard recombinant DNA techniques well known in the art are employed to generate the transgene vectors and expression constructs. Such expression constructs comprise nucleic acid sequences encoding a protein of interest operably linked to regulatory elements necessary for expression of the polypeptide in the host cell.

In a preferred embodiment promoters capable of expressing the polypeptide in specific tissues are employed. For example, to produce a recombinant protein in the milk of a transgenic animal, expression vectors are constructed by fusing the gene encoding the recombinant protein to regulatory elements of a milk specific protein such as beta-casein, beta-lactoglobulin, whey acidic protein and alph-lactalbumin. (Pollock, 1999). The expression vector is then microinjected into a one-cell embryo, and the injected embryo is implanted into a suitable surrogate animal. The resulting transgenic animals can produce the recombinant protein in their milk. Large-scale production of monoclonal antibodies can be obtained by generating transgenic dairy animals, such as goats, capable of producing antibodies in their milk. (see, e.g., Meade et al., 1998; Velander et al., 2002).

Several methods are well known in the art to produce transgenic animals. These include, but are not limited to, introduction DNA into embryos by microinjection into pronuclei, introduction of totipotent or pluripotent stem cells transformed with the DNA into embryos and infection of embryos with retroviral vectors. The embryos harboring transgene are then allowed to develop into mature transgenic animals. Methods for obtaining transgenic animals are well known in the art (e.g., Houdebine, 1997; Hogan et al., 1986; Krimpenfort et al., 1991; Palmiter et al., 1985; Kraemer et al., 1985; Hammer et al., 1985; Wagner et al., 1992; Krimpenfort et al., 1992; Jänne et al., 1992; Brem et al., 1993; and Clark et al., 1995). Transgenic animals can also be generated using methods of nuclear transfer or cloning using embryonic or adult cell lines (e.g., Campbell et al., 1996; and Wilmut et al., 1997). Further a technique utilizing cytoplasmic injection of DNA may be employed (Page et al., 1996).

II. Production of Proteins in Animals

As discussed above, proteins can be expressed in transgenic cells in vitro and in vivo. Cells capable of expressing a protein of interest may secrete protein into the culture medium. Alternatively, proteins can be expressed as intracellular proteins.

Transgenic animals may be generated such that they express a polypeptide of interest into surrounding tissues or body fluids. Preferably, such bodily fluids include serum, plasma, whole blood, urine and milk. In a most preferred embodiment, the polypeptide of interest is expressed in the milk of a transgenic animal. In addition, the most preferred embodiment comprises a polypeptide expressed in the milk of a transgenic goat.

Any polypeptide of interest can be expressed from a transgene. Such polypeptides include but are not limited to, enymes (e.g., ribonuclease, trypsin), transport proteins (e.g., hemoglobin, serum albumin), nutrient and storage proteins (e.g., ovalbumin, casein), contractile or motile proteins (e.g., actin, myosin), structural proteins (e.g., collagen, fibrin, elastin), defense proteins (e.g., antibodies, fibrinogen, thrombin), and regulatory proteins (e.g., cytokines, receptors, insulin, growth hormone, repressors). Preferred proteins include antibodies, especially therapeutic antibodies. More preferably, the antibodies are fully human, humanized, or chimeric constructs. In the most preferred embodiment of the invention, the protein of interest is an anti-Tumor Necrosis Factor (TNF) antibody, such as D2E7, as described by Salfeld et al. (2000).

III. Collection of Bodily Fluids

Various bodily fluids from a transgenic animal expressing a polypeptide of interest may be collected. Subject to practitioner preference, the method of collection and treatment of the bodily fluid will also depend upon the animal and type of fluid collected. One skilled in the art will appreciate that numerous techniques are available to effect and to facilitate the isolation of different types of bodily fluids. For example, blood can be isolated from an animal by exsanguination. Milk may be obtained from a lactating transgenic animal by mechanical or other extraction means. Such techniques are commonly used in the dairy industry (see, e.g., McBurney et al., 1964; and Velander et al., 1992).

IV. Polypeptide Modifications

Recombinant polypeptides expressed in cell culture or animal production systems can undergo post-translational modifications, post-secretional modifications, and other modifications. The present invention provides a method of preventing and reducing modification of polypeptides in solution.

The nature of polypeptide modifications include the addition of undesirable radical groups or side chains to a polypeptide of interest. Such modifications include but are not limited to, glycosyl, glucuronidyl, peptidyl, phosphoryl, sulphuryl, farnesyl, acyl, or maleuryl group additions to the polypeptide of interest.

In one embodiment, the invention pertains to polypeptides in milk which undergo modification when stored for a period of time prior to separation. One type of modification, called acidic modification of a polypeptide, is revealed in the present invention. Acidic modification of a polypeptide can be detected using a weak cation exchange column (WCX-10) as described herein. Further analysis reveals that such acidic modification of a polypeptide can be caused by the addition of one or more maleuryl groups.

V. Preparation of a Polypeptide-Containing Solution

In order to obtain sufficient quantities of polypeptide for subsequent use, large quantities of polypeptide-containing solution may be required. Under these circumstances it may be necessary to collect and to store the solution containing the polypeptide of interest (e.g., bodily fluid) until sufficient quantities of the fluid has been obtained for efficient isolation of the polypeptide. Storage of the protein-containing solution is understood as meaning any storage of solution (e.g., bodily solution) containing a protein of interest, regardless of the volumetric amount, the time period of storage, the temperature of storage conditions, the addition of other agents, or other appropriate treatment conditions or parameters.

Polypeptide-containing solutions are often stored at temperatures below room temperature (less than about 28° C.), and typically at or below about 4° C., to minimize protein degradation. It is also well known in the art that the addition of agents, such as sodium azide and EDTA, may be made to prevent or to slow, for example, bacterial growth.

The method of the present invention is independent of such additional parameters and treatment conditions. The method may include, for example, storage for a period of time (at any practitioner-selected temperature) before and/or after the acid treatment of the present invention. In addition, the method of the present invention may comprise further treatment conditions, such as the addition of additional agents and/or preparative compositions. Such additional treatment methods are not necessary to practice the present invention, however.

Preferably, the method of the present invention comprises storing a solution containing a X polypeptide of interest below room temperature. In a preferred embodiment, the storage temperature can range from about 15° C. to about −80° C. In a more preferred embodiment the storage temperature can range from about 4° C. to about −20° C.

VI. Acid Treatment to Reduce or to Prevent Modification of Polypeptide

The present invention is directed to reducing or to preventing modification of a polypeptide of interest in solution by adding acid to the polypeptide-containing solution. In a specific embodiment, acid is added to the solution. Such acids include weak and strong acids capable of reducing the pH of the polypeptide-containing solution. One skilled in the art will recognize that pH can be measured using any of a number of standard techniques, assays, and instruments known in the art.

The amount of acid to be added to the polypeptide-containing solution is an amount sufficient to achieve the appropriate pH. The appropriate pH is the pH at which the modification of the polypeptide is prevented or reduced and is dependent on the chemical characteristics of the polypeptide of interest and of the accompanying solution. Determination of the appropriate pH for a given polypeptide of interest in a given solution is practitioner-determined following protocols known to persons of ordinary skill in the art. It is also noted that the low pH conditions of the present acid treatment also facilitate any “viral kill step” known to be desirable for the preparation of solutions from biological samples.

Typically, amount of acid added to a polypeptide-containing solution (e.g., milk) is that amount sufficient to obtain a pH of about pH 7.0 to about pH 1.0. Preferably, sufficient acid is added such that the pH of the polypeptide-containing solution is about pH 5.0 to about pH 2.0. Even more preferably, sufficient acid is added such that the pH of the polypeptide-containing solution is about pH 4.0 to about pH 3.0. Most preferably, sufficient acid is added such that the pH of the solution or bodily fluid is about pH 3.5 to about pH 3.0.

Strong or weak acids are useful for the practice of the present invention. Preferably, the strong or weak acid is selected from the group of acids consisting of acetic acid, citric acid, formic acid, and hydrochloric acid. More preferably the acid is citric acid. In a most preferred embodiment, the acid is 2.5M citric acid.

VII. Storage Temperature

After the acid treatment discussed above, polypeptide-containing solutions may be immediately used for further purification process, or (also as discussed earlier) may be stored for a period of time. In one preferred embodiment of the present invention, the acid treated polypeptide-containing solution is stored at a temperature below room temperature. In one preferred embodiment of the invention, the temperature at which the acid treated polypeptide-containing solution is stored is about 4° C. to about −80° C. In a more preferred embodiment, the temperature is about 0° C. to about −70° C. More preferably, the temperature is about −1° C. to about −50° C. Most preferably, the temperature is about −15° C. to about −30° C. Ideally, the temperature at which the milk is stored is about −20° C.

In one embodiment, the invention provides a method for reducing or preventing modification of polypeptides in milk from transgenic animals. In a preferred embodiment, acid is added to milk containing a polypeptide to reduce or to prevent modification of the polypeptide. The acid treated milk is then stored at a temperature below room temperature. The polypeptide may later be isolated from the acidified milk.

VIII. Isolation of Protein from a Polypeptide-Containing Solution

According to the present invention, an isolated protein, is a protein that is substantially pure. Proteins of the present invention may be purified using a variety of standard protein purification techniques, such as, but not limited to, precipitation, centrifugation, filtration, affinity chromatography, immunoaffinity chromatography, ion exchange chromatography, electrophoresis, hydrophobic interaction chromatography, gel filtration chromatography, reverse phase chromatography, concanavalin A chromatography, chromatofocusing and differential solubilization.

The procedure for isolating a polypeptide from solution will depend on the nature of the starting material containing the polypeptide to be isolated, and, the nature of the polypeptide itself. A wide variety of isolation techniques and procedures are known and available to persons skilled in the art. Selection of any particular isolation technique is determined by practitioner preference.

In a preferred embodiment, recombinant proteins are expressed in milk. In a more preferred embodiment the recombinant proteins are expressed as whey proteins and (typically) isolated using, for example, Ultra filtration. Milk solids, lipids, and other milk proteins are separated from the polypeptide of interest. The polypeptide containing ‘permeate’ is subjected to concentration and other chromatographic steps further to isolate the polypeptide. Specifically preferred protocols are exemplified in the Examples section infra.

In a most preferred embodiment, the invention provides a method of preventing and reducing modification of D2E7 in milk obtained from a transgenic goat, comprising the steps of: a) providing transgenic goat milk containing D2E7; b) adding 2.5 M citric acid to said milk in an amount sufficient to obtain a pH of said milk of about pH 3.0 to about pH 3.5; c) storing said milk at a temperature below room temperature; and d) isolating said D2E7 from said milk.

IX. Stabilized Polypeptides

The present invention can be used to stabilize proteins produced in transgenic animals. These proteins include but are not limited to hormones such as insulin, and growth hormone; cytokines such as interleukins, tumor necrosis factor, epidermal growth factor, and platelet derived growth factor; immunoproteins such as antibodies, fusion proteins, and chimeric proteins; protein components found in blood clotting cascade such as Factor VIII; enzymes, and carrier proteins.

One aspect of the present invention is directed to a polypeptide isolated according to the foregoing method.

The present invention incorporates by reference in their entirety techniques well known in the field of molecular biology. These techniques include, but are not limited to, techniques described in the following publications:

Ausubel, F. M. et al. eds., Short Protocols In Molecular Biology (4th Ed. 1999) John Wiley & Sons, NY. (ISBN 0-471-32938-X).

Fink & Guthrie eds., Guide to Yeast Genetics and Molecular Biology (1991) Academic Press, Boston. (ISBN 0-12-182095-5).

Hogan et al., Manipulating The Mouse Embryo (1986) Cold Spring Harbor Press.

Houdebine, Transgenic Animal Generation and Use (1997) Harwood Academic Press.

Kay et al., Phage Display of Peptides and Proteins: A Laboratory Manual (1996) Academic Press, San Diego.

Kraemer et al., Genetic Manipulation of the Early Mammalian Embryo (1985) Cold Spring Harbor Laboratory Press.

Lu and Weiner eds., Cloning and Expression Vectors for Gene Function Analysis (2001) BioTechniques Press. Westborough, Mass. 298 pp. (ISBN 1-881299-21-X).

Old, R. W. & S. B. Primrose, Principles of Gene Manipulation: An Introduction To Genetic Engineering (3d Ed. 1985) Blackwell Scientific Publications, Boston. Studies in Microbiology; V.2:409 pp. (ISBN 0-632-01318-4).

Robinson ed., Sustained and Controlled Release Drug Delivery Systems (1978) Marcel Dekker, Inc., NY.

Sambrook, J. et al. eds., Molecular Cloning: A Laboratory Manual (2d Ed. 1989) Cold Spring Harbor Laboratory Press, NY. Vols. 1-3. (ISBN 0-87969-309-6).

Stewart and Young, Solid Phase Peptide Synthesis (2d. Ed. 1984) Pierce Chemical Co.

Winnacker, E. L. From Genes To Clones: Introduction To Gene Technology (1987) VCH Publishers, NY (translated by Horst Ibelgaufts). 634 pp. (ISBN 0-89573-614-4).

It will be readily apparent to those skilled in the art that other suitable modifications and adaptations of the methods of the invention described herein are obvious and may be made using suitable equivalents without departing from the scope of the invention or the embodiments disclosed herein. Having now described the present invention in detail, the same will be more clearly understood by reference to the following examples, which are included for purposes of illustration only and are not intended to be limiting of the invention.

### EXAMPLE 1

### Generation of a Transgenic Goat Producing D2E7

D2E7 is a fully human IgG1 antibody (Ab) to tumor necrosis factor alpha (TNFα) (see Salfeld et al, 2000 and 2001, incorporated by reference). Expression vectors were constructed by placing the genes for D2E7 antibody under the regulation of a milk specific promoter, the betacasein promoter DNA (Boss et al., 1984; Zala, 1995; Bebbington and Houdebine, 1994; Houdebine, 1995; and Echelard, 1996). Transgene expression vectors were microinjected into fertilized eggs and transferred into recipient female goats. Offspring were tested for presence of the transgene. Transgenic goats were mated. The resulting (F₁) transgenic females were capable of producing milk containing recombinant goat D2E7 (G-D2E7).

### EXAMPLE 2

### Transgenic Goat Milk Collection and Storage: Without Acid Treatment

Transgenic G-D2E7 goats produced according to Example 1, were milked. Immediately upon collection, the milk was frozen in 1L and 2L bottles at −20° C. Collected milk was transported in dry ice and subsequently placed in a −80° C. freezer. Large volumes of milk (2-20 L) were collected for later G-D2E7 purification.

### EXAMPLE 3

### Isolation of Recombinant D2E7 from Untreated Transgenic Goat Milk

Transgenic goat milk containing D2E7 obtained according to Example 2 was thawed, at room temperature, for approximately 15 hours in batches of 10×1L bottles. Ten 1L aliquots of milk were pooled and diluted 10% with 0.5 M EDTA, pH 8.0. The sample was clarified over a 500 K Omega UF cassette (Pall Filtron Corporation, Northborough, Mass.) following manufacturer's instructions. D2E7 was passed through the cassette into the ‘permeate’, milk solids, lipids and high molecular weight milk proteins were retained in the concentrate. The milk was concentrated 5 fold, and washed with 4 diafiltration volumes of 0.02 M EDTA, pH 8.0. The clarified milk was then run over a High S cation-exchange capture column (Bio-Rad Laboratories, Hercules, Calif.) following manufacturer's instructions. The High S eluate was then run over a virus removal filter, Ultipor DV50 (Pall Filtron Corporation, Northborough, Mass.), and subsequently on an anion exchange column, Q Sepharose FF (Amersham Biosciences, Piscataway, N.J.) run in flow-through mode. This process was followed by a run on hydrophobic interaction column, Phenyl Sepharose FF (Amersham Biosciences, Piscataway, N.J.) following manufacturer's instructions. The Phenyl Sepharose eluate was concentrated and exchanged with PBS buffer over an A1Y10 cartridge (Millipore, Bedford, Mass.) following manufacturer's instructions.

### EXAMPLE 4

### Purification of Recombinant D2E7 from Untreated Transgenic Goat Milk

G-D2E7, isolated in the eluate prepared according to Example 3, was purified using rProtein A affinity chromatography following manufacturer's instructions. G-D2E7 adsorbed to rProtein A Sepharose Fast Flow (Amersham Pharmacia Biotech, Piscataway, N.J.), while contaminants flowed through. The rProtein A Sepharose FF resin was loaded at 25 g/L resin. The column was then washed with equilibration buffer, for a minimum of 15 column volumes, to ensure complete separation of lactoperoxidase from G-D2E7. Product was eluted with 20 mM NaAcetate, 40 mM NaCl, pH 3.5. The column eluate was collected from 5% to 5% deflection (i.e. from initial 5% over baseline point to subsequent 5% over baseline point) and the pH was adjusted to neutral with 200 mM Trolamine, 40 mM NaCl, pH 8.5. The pH adjusted eluate, containing G-D2E7, was filtered through a 0.2 μM polyethersulfone membrane ACRODISC (Pall Corporation, NY), and concentrated using regenerated cellulose acetate CENTRIPREP YM30 (Millipore, Bedford, Mass.). G-D2E7 concentration was determined by the spectroscopy at UV 280 nm, and calculated using the molar absorbance of 1.39 mL/mg.

### EXAMPLE 5

### G-D2E7 Modification: Detection of Acidic Peaks Using a WCX-10 Assay

Because heterogeneity of antibodies may arise due to C-termini, N-termini, carbohydrates, deamidation and protein aggregation and degradation, a WCX-10 assay was developed to identify D2E7 isoforms purified from Chinese Hamster ovary (CHO) cells (Santora et al., 1999). This assay was used to analyze D2E7 isolated and purified from transgenic goat milk.

G-D2E7 antibody purified according to Example 4 was diluted with HPLC grade water to a concentration of 1.0 mg/mL for HPLC analysis. Different isoforms of G-D2E7 were separated on a WCX-10 column with a WCX-10 guard column (Dionex Corporation, Sunnyvale, Calif.) on a Shimadzu HPLC, Model 10A (Shimadzu Scientific Instruments, Inc., Columbia, Md.) following manufacturer's instructions. Column oven temperature was set at 30° C., and UV detection at 280 nm was used to monitor the protein. Buffer A (A) was 10 mM NaH₂PO₄, pH 7.5; buffer B (B) was 10 mM NaH₂PO₄ and 500 mM NaCl, pH 5.5. The flow rate was 1.0 mL/min, and the injection amount was 100 μg. Linear gradient conditions were from 6% B to 16% B in 20 minutes; followed by a 100% B wash of the column for 10 minutes; followed by 6% B equilibration for another 6 minutes.

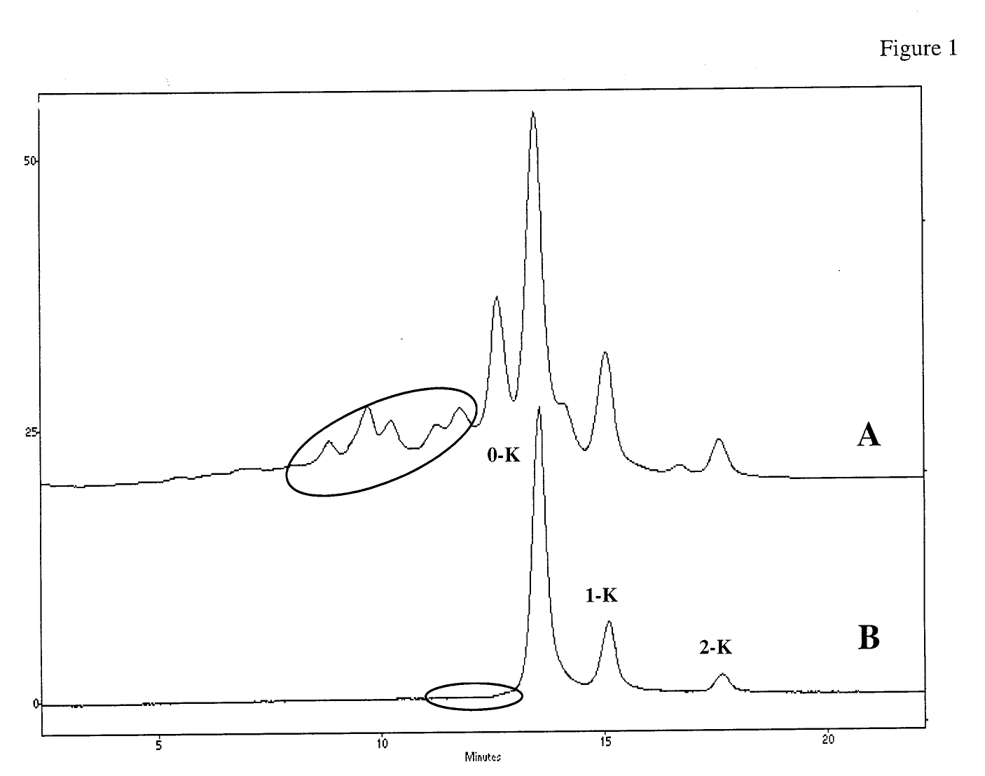

G-D2E7 derived from transgenic goats was compared with D2E7, expressed in and isolated from CHO cells (CHO D2E7) (see Salfeld et al, 2000 and 2001). Results indicate that G-D2E7 contained 42% acidic isoforms, whereas CHO D2E7 contained only 10% acidic isoforms (FIG. 1). Because D2E7 isoforms have been previously characterized (Santora et al., 1999), three peaks from 2E7 or G-D2E7 are known to be O-Lysine, 1-Lysine and 2-Lysine isoforms on the heavy chain C-termini (as labeled in FIG. 1).

Oligosaccharide analysis indicated that G-D2E7 had sialic acids on oligosaccharides. Therefore, some chromatographic peaks of G-D2E7 were due to sialic acid isoforms. Enzymatic treatment was performed to confirm these results. Treatment with the protease carboxypeptidase B (CPB), resulted in the collapse of all of the heavy chain C-terminal Lys isoform peaks into one 0-Lys peak. Treatment with sialidase enzyme removed sialic acids from the oligosaccharide residues, and some of the sialic acid isoform peaks disappeared. Some unknown acidic peaks of G-D2E7 remained, however (see FIG. 1).

### EXAMPLE 6

### D2E7-TNF™ Binding Assay

The ability of G-D2E7 isoforms, purified according to Example 4, to bind TNFα was tested. Purified G-D2E7 (1.0 mg/mL), TNFα (0.2 mg/mL) and D2E7-TNFα complexes were separated on a Dionex weak cation exchange column (WCX-10) with a WCX-10 guard column (Dionex Corporation, Sunnyvale, Calif.) on a Shimadzu HPLC, Model 10A (Shimadzu Scientific Instruments, Inc., Columbia, Md.) following manufacturer's instructions. Column oven temperature was set at 30° C. and LW detection at 280 and 214 nm were used to monitor the proteins. Buffer A (A) was 10 mM phosphate, pH 7.5, and buffer B (B) was 10 mM phosphate and 500 mM NaCl, pH 5.5. The flow rate was 1.0 mL/min, and the injection volume was 100 μL. Linear gradient conditions were from 3% B to 16% B in 20 minutes; changed to 16% to 50% in another 20 minutes; followed by a 100% B wash of the column for 6 minutes, and followed by 3% B equilibration for another 5 minutes.

G-D2E7 (1.0 mg/ml) and TNF-α (0.4 mg/ml) were mixed at room temperature (25° C.) for 30 seconds. The mixture was injected onto the WCX-10 column, and G-D2E7, TNF-α and G-D2E7·TNF-α complexes separated.

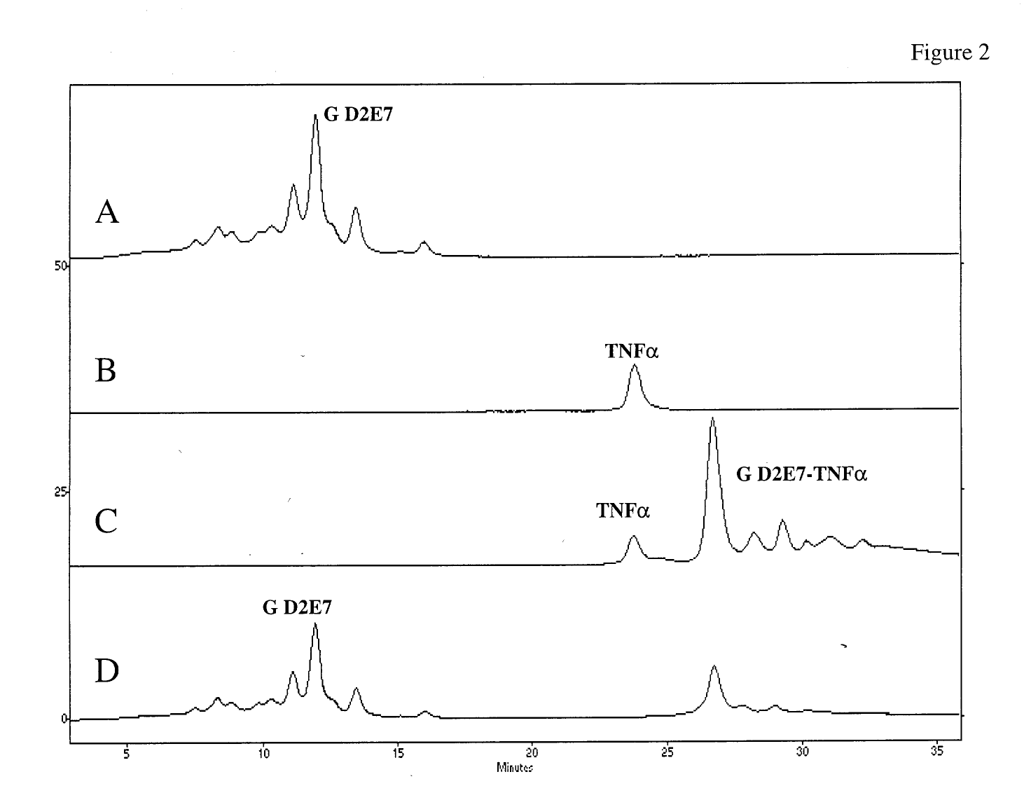

G-D2E7 was heterogeneous, producing four peaks due to charge heterogeneity of the heavy chain C-terminal Lys variants and acidic peaks (FIG. 2A). G-D2E7 eluted before TNF-α, which eluted at about 24 minutes (FIG. 2B).

All of the acidic peak variants were able to bind TNFα, equally well. G-D2E7-TNFαa complexes eluted after 26 minutes (FIGS. 2C and D). All of the G-D2E7 peaks disappeared after mixing G-D2E7 with excess amounts of TNFα (FIG. 2C); in other words, a flat baseline was observed where the G-D2E7 isoforms normally eluted. This indicates that all of the G-D2E7 isoforms specifically bound TNFα in the presence of excess TNFα and were not impurities. Relative percentage of unbound G-D2E7 isoforms shows that all of the antibody isoforms have similar affinities, or that charged G-D2E7 variants bind TNFα equally well.

### EXAMPLE 7

### Molecular Weight Analysis of G-D2E7 Heavy and Light Chains Using RP/C4/HPLC

G-D2E7, isolated according to Example 4, was broken down into two fragments, Fab and Fc, using the enzyme papain and Fab and Fc fragments were separated using a Protein A column following procedures well known in the art (Fc bound to the column whereas the Fab flowed through).

The molecular weights of G-D2E7 Fc and Fab fragments were determined by HPLC/MS after Protein A separation. Fc fragments were deglycosylated by PNGase F and reduced by DTT. Molecular weight of the Fc fragment was determined using mass spectroscopy. No modification was observed on the Fc fragments.

Fab fragments were separated using a WCX-10 column. Several acidic peaks were observed. Fab acidic peaks had similar profiles compared to the full length G-D2E7 acidic peaks indicating GD2E7 acidic peaks are primarily from the Fab region. All of these peaks were fractionated and analyzed using HPLC/MS.

G-D2E7 Fab isoforms or fractions were reduced by a 1.0 M DTT solution. A Vydac protein C4 column (CN 214TP5115, The Nest Group, Inc., Southboro, Mass.) was used to separate heavy and light chains of D2E7 Fab. Buffer A was 0.02% trifluoroacetic acid (TFA; PIERCE, CN. 53102)+0.08% formic acid (FA; Sigma, F0507)+0.1% acetonitrile (ACN; Burdick & Jackson, CN. 0154)+99.8% HPLC-H₂O. Buffer B was 0.02% TFA+0.08% FA+0.1% HPLC-H₂O+99.8% ACN. The flow rate was 0.05 mL/min and the injection volume was 5.0 μL for 0.1 mg/mL of the samples. The column oven was set at 30° C., and separation conditions were as in Table 1a.

TABLE 1a
HPLC Gradients for Reduced Fab Analysis Using a C4 Column

| | | | | | | | | |
|---|---|---|---|---|---|---|---|---|
| Time (min) | 0 | 5 | 6 | 30 | 31 | 36 | 37 | 45 |
| B % | 5 | 10 | 30 | 50 | 80 | 80 | 5 | 5 |

The same buffers A and B were used with both C4 and C18 columns for the separation of either proteins or peptides

Fraction 7 (F7) was the major peak, which represented 70% of the total protein, including all of the 0-Lys, 1-Lys and 2-Lys G Ab1 isoforms. Fraction 2 (F2) represented 5% of the total protein and Fraction 5 (F5) represented only 1% of the total protein. Since the Fab disulfide bonds (S—S bonds) were reduced by DTT, the dissociated Fab light chain (LC) and heavy chain (HC′) fragments were separated by the C4 column and determined by MS respectively. Three typical results were obtained for each fraction of the Fab isoforms when measuring the MWs of the Fab LC and HC′ fragments. There was no modification on either the HC′ or the LC for F7. The theoretical MW of the LC is 23,412 Da, and the measurement was 23,411±1 Da. The theoretical MW of the HC′is 24,279 Da and the measurement was 24,278±1 Da. The MWs of the LC and HC′ for F2 after deconvolution showed that the MW measurements were 23,552±1 Da for the modified LC peak (mLC), 23,411±1 Da for another small LC peak, and 24,280±1 Da for the HC′. The MWs of the LC and HC for F5 after deconvolution showed that the MW measurements were 23,552±1 Da for the mLC peak and 23,411±1 Da for the LC peak. The MW measurements were 24,280± 1 Da for the HC peak and 24,419±1 Da for another modified HC′ peak (mHC′). The rest of the reduced Fab fractions had different ratios of the rnLC to the mHC′, respectively.

These results demonstrated that: i). F7 was the standard Fab; ii). F2 was the Fab with modified mLC, which had an additional mass of 141 Da on the LC, and the standard HC′; and iii). F5 was the Fab with a partially modified mLC and mHC′, which had additional mass of 140 Da derivative on both the LC and HC′.

### EXAMPLE 8

### G-D2E7 Peptide sequence analysis using RP/C18/HPLC and Q-TOF

G-D2E7, isolated according to Example 4, was digested with trypsin and the trypsin-digested peptides separated on a Vydac protein & peptide C18 column (CN 218TP51, The Nest Group, Inc., Southboro, Mass.). Buffer A was 0.02% TFA+0.08% FA+0.1% ACN+99.8% HPLC-H₂O. Buffer B was 0.02% TFA+0.08% FA+0.1% HPLC-H20+99.8% ACN. The flow rate was 0.05 mL/min and the injection volume was 20 μL for 0.1 mg/mL of total peptide. The column oven was set at 30° C. and separation conditions were as in Table 1b.

TABLE 1b
HPLC Gradients for Peptide Analysis Using a C18 Column

| | | | | | | | | |
|---|---|---|---|---|---|---|---|---|
| Time (min) | 0 | 5 | 145 | 155 | 160 | 172 | 175 | 190 |
| B % | 0 | 5 | 40 | 50 | 80 | 80 | 0 | 0 |

Note:
The same buffers A and B were used with both C4 and C18 columns for the separation of either proteins or peptides

Under these conditions complete separation of G D2E7 tryptic peptides was achieved as detected by the UV detector at 214 and 280 nm. The peptides separated from the HPLC instrument flowed directly into the MS source.

G-D2E7 trypsin-digested peptides, separated using RP/C18/HPLC, were then analyzed using a quadrupole orthogonal acceleration time of flight (Q-TOF) mass spectrometer (Micromass, Beverly, Mass.), with a standard Z-spray source fitted metal electrospray probe (see Larsen and McEwen, 1998). Needle voltage was 3200V, and the cone voltage was 50V. The source block and the desolvation temperature were 90° C. and 110C, respectively. Rates of desolvation gas and nebuliser gas were 250 L/h and 4 L/h. All samples were continuously infused through the electrospray probe after HPLC separation. The scan duration and the interscan delay were 0.90 and 0.10 seconds (secs) for all experiments. All data were acquired based on survey scans with the automated MS to MS/MS function switching.

To obtain optimum fragmentation of precursor ions selected for MS/MS, a collision energy profile was performed. This profile applied 30% of the collision energy for the m/z range from 200 Da to 1000 Da; 35% of the collision energy for the m/z range from 1000 to 2000 Da; 40% of collision energy for the m/z range from 1500 Da to 2500 Da, and 45% of collision energy for the m/z range from 2000 Da up to 4000 Da. MassLynx software was used for peptide analysis.

All tryptic peptides were analyzed by C18/MS/MS. By checking the MWs of peptides, based on the known sequence, an additional mass of 140 Da was detected on the LC N-terminal peptide for the modified Fab fraction. The standard Fab peptide sample was used as a control to avoid any artifacts due to the sample preparation and during the ionization process in the gas phase. The measured MW of the standard peptide was 1878.548 Da (theoretical=1879.035 Da), or the doubly charged ion peak of 940.274. The modified LC N-terminal peptide has the MW of 2018.558 Da, or a doubly charged ion peak of 1010.279. Therefore, the additional mass of the modified peptide (the derivative) was Δm=140.01±0.01 Da.

The modified and unmodified peptides derived from the Fab fraction were further analyzed using collision-induced dissociation (CID) mass spectrometry (CID/MS/MS) using standard protocols as described in Larsen and McEwen, (1998). The sequence of the unmodified peptide was determined and analysis of the modified peptide indicated that the N-terminal Aspartic acid was modified by the addition of a 140 Da derivative.

### EXAMPLE 9

### Peptide Modification Analysis

The Modified G-D2E7 antibody peptide was analyzed following acetylation protocols, HPLC/MSIMS, and Q-TOF/MS/MS (Micromass, Beverly, Mass.) (see Larsen and McEwen, 1998) known in the art to locate the 140±1 Da modification, detected in Example 8, on the N-termini of the modified peptide.

The standard peptide was acetylated by (CH₃CO)₂O, and the mass changed to 1920 Da (1878+42=1920). In addition, all b ions exhibited an additional 42 Da, due to acetylation but y ions exhibited no change for the standard N-terminal peptide by HPLC/MS/MS. The mass of the modified peptide, however, did not change after acetylation and remained 2020 Da. These results indicated that there was no acetylation on this peptide and that the N-terminus was blocked by post-secretional modification on the N-terminus of Asp.

All b ions from the modified peptide exhibited an additional mass of 140 Da, due to the post-secretional modification, whereas all y ions exhibited no change for the modified N-terminal peptide by HPLC/MS/MS. The acetylation method results confirm that the 140 Da peptide modification is located on the N-termini of the modified peptide, and that there were no free amino termini on the modification. It was confirmed that the post-secretional modification was on the amino acid aspartate (not isoleucine). Further elemental composition analysis results from the QTOF revealed that the unknown derivative was a maleuric acidic modification.

### EXAMPLE 10

### Temporal Effects On Polypeptide Modification

To demonstrate that, left untreated, polypeptide modification in solution increases over time, post secretional modification of G-D2E7 modification in milk was analyzed over time.

Small aliquots (50 ml) of G-D2E7 transgenic milk, collected according to Example 2, were taken from a −80° C. freezer and immediately placed in a water bath, set at 37° C., for 15 minutes. The samples were then purified by rProtein A, concentrated, and run on the Cation-exchange liquid chromatography (CIEX; see Example 4). No acidic peaks were observed by the WCX-10 assay (see Example 5).

Because large-scale protein filtration and purification processes require longer periods of time to perform, time course experiments were performed to determine the extent of protein modification over time.

Bottles of transgenic milk (1L), collected according to Example 2, were thawed at 4° C., at its natural pH of about 6.5, for a time period of 65 and 96 hours. To prevent bacterial growth, 0.1% sodium azide was added in the milk for a final concentration. The samples were then purified by rProtein A, concentrated, run on the CIEX (Example 4), and analyzed using the WCX-10 assay (see Example 5).

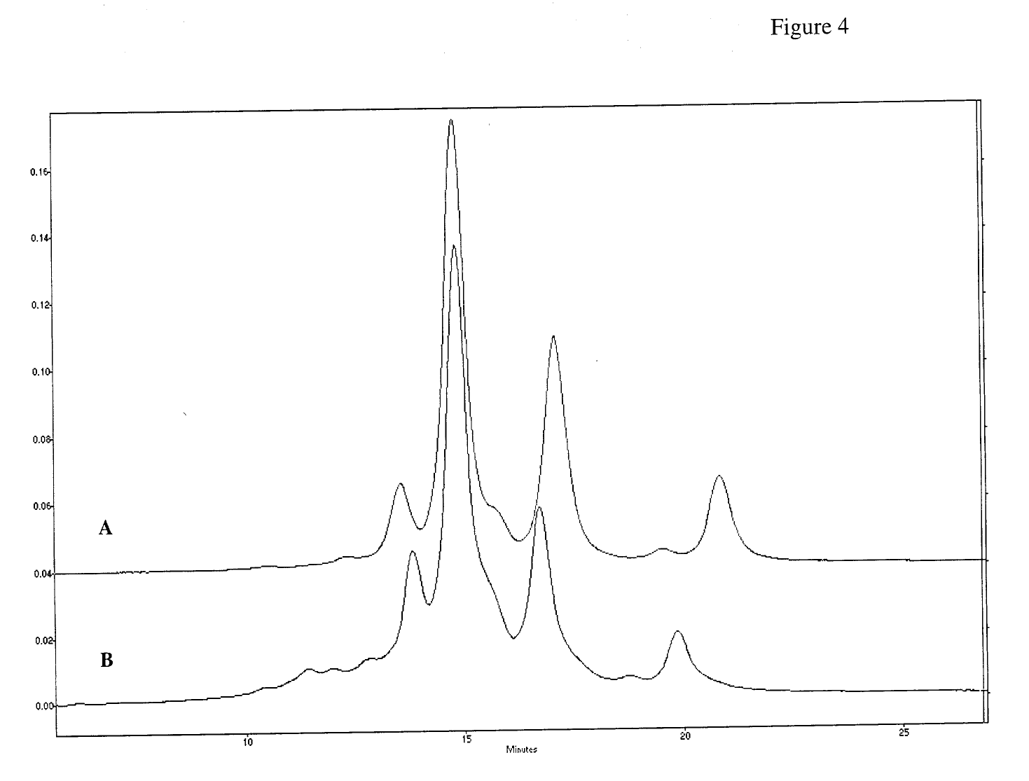

At 65 hours, the acid peak level measured 3% of the total area; while at 96 hours, the acidic peak level rose to 15% acidic isoforms (FIG. 4).

These results demonstrate that, left untreated, the amount polypeptide modification in solution increases over time.

### EXAMPLE 11

### Temperature and pH Effects on Polypeptide Modification

To demonstrate that polypeptide modification in solution increases with increasing temperature and/or pH, post secretional modification of G-D2E7 modification in milk was analyzed a range of temperature and pH conditions.

Milk aliquots (1˜2L) of G-D2E7 transgenic milk, collected according to Example 2, were taken from a −80° C. freezer and thawed at 4° C., for 48 hours. Sodium azide was added to a final concentration of 0.1% to prevent bacterial growth. One half of the milk (500 ml) was adjusted to pH 3.0, adding 2.5 M citric acid; the remaining half of the milk (500 ml) was left untreated. The natural pH of milk is about pH 6.5 to 7.0. Each milk sample was divided equally and subsequently incubated at room temperature (18-23° C.) and 37° C. for 96 hours. Test and control samples were analyzed daily using the WCX-10 assay (see Example 5). Acid peaks were integrated to obtain relative percentage values.

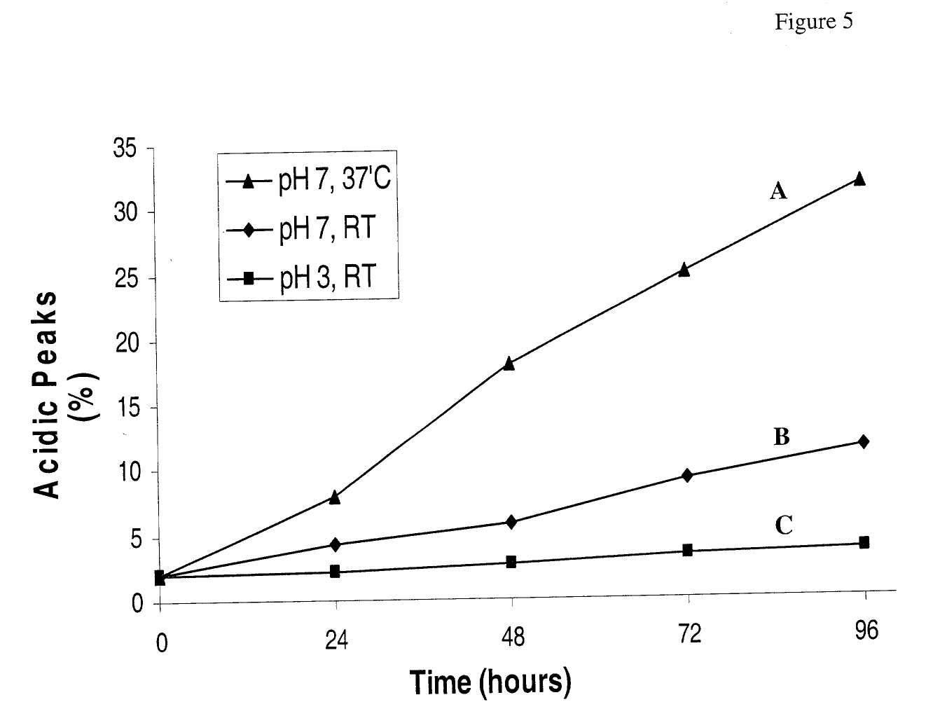

As previously demonstrated (Example 10), in milk samples left at neutral pH, acidic peak percentages of G-D2E7 steadily increased over time. In samples incubated at elevated temperature (37° C.), acidic peak percentages of G-D2E7 markedly increased over the 96 hour period (to 30% acidic peaks). Acid-treated (pH 3.0) transgenic G-D2E7 milk remained stable, however (see FIG. 5).

It was further demonstrated that when the pH of transgenic milk containing G-D2E7 was raised to pH 9, more chromatographic peaks were observed, and the protein was less stable than when the protein was left at neutral pH (data not shown).

These results demonstrate that under conditions of elevated temperature and pH the amount polypeptide modification in solution increases over time. Acid treatment according to the present invention, however, prevents polypeptide modification, even at room temperature.

### EXAMPLE 12

### Post-Secretional Modifications of Other Proteins in Milk

To demonstrate that a variety of proteins (other than G-D2E7) are susceptible to post-secretional modification in solution, different proteins, placed in milk solution, were analyzed for subsequent protein modification.

D2E7, produced and isolated from CHO cell line (CHO-derived D2E7), and an anti-1L-12 antibody (J695), produced and isolated from a CHO cell line (CHO-derived anti 1L-12 antibody), were separately spiked into non-transgenic milk at a concentration of 2 mg/mL. Each test sample was incubated in non-transgenic goat milk at 37° C. for 66 hours. The samples were purified over rProtein A and assayed by WCX-10 as previously described.

Untreated CHO-derived D2E7 exhibited an increase in acidic peak formation, from 10% to 27%, analyzed using HPLC with a WCX-10 column. These CHO-derived D2E7 acidic peaks were fractionated and analyzed by HPLC/MS. The modifications on CH0-D2E7 were identical to those observed with untreated G-D2E7. Similarly, untreated CHO-derived anti 1L-12 antibody exhibited an increase in acidic peak formation, from 9% to 17%, analyzed using HPLC with a WCX-10 column.

The results demonstrate that protein modification in milk solution is a generic problem, not unique to the G-D2E7 antibody.

### EXAMPLE 13

### Peptide Modification Reduction

To demonstrate the ability of a low pH buffer to reduce or to prevent peptide modifications in solution, G-D2E7 peptides were quenched by IM formic acid (FA) resulting in the cleavage of the modification from the peptide.

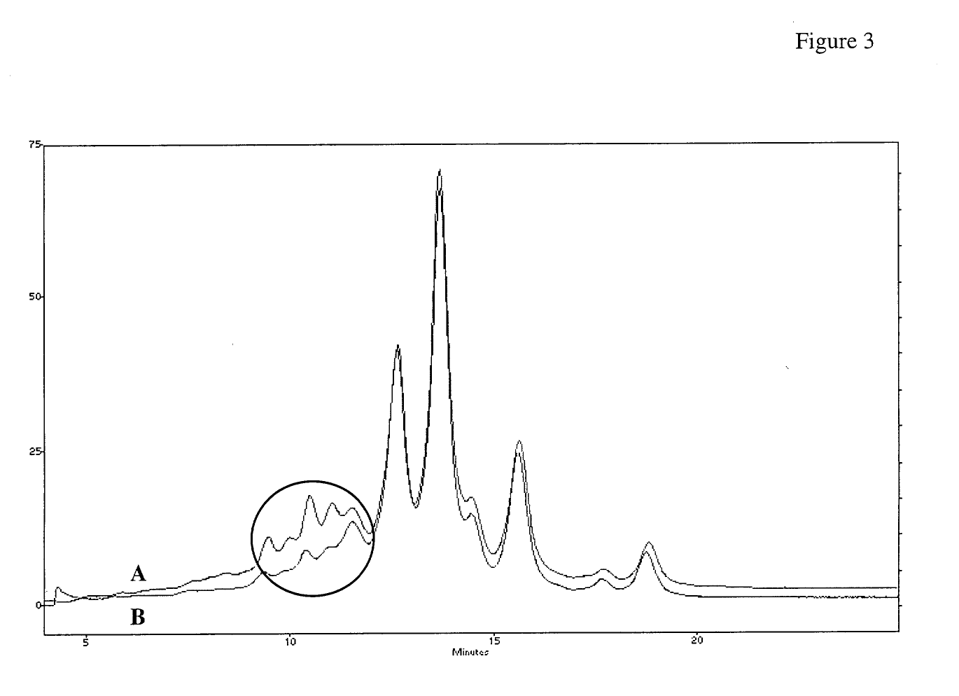

FIG. 3 represents a chromatographic comparison of G-D2E7 acidic peaks before and after formic acid treatment by CIEX. Chromatogram A illustrates G-D2E7 without formic acid treatment, containing 42% acidic peaks eluting at 10 minutes. Chromatogram B illustrates G-D2E7 after formic acid treatment.

As a further demonstration, G-D2E7 peptides were dialyzed into 50 mM NH4HCO3+FA, pH3.0 and pH3.25 buffers over time to remove the peptide modification. The results confirm that the amount of acidic peaks in the D2E7 samples, due to the post-secretional modification, were decreased significantly with formic acid treatment, and demonstrate that acid treatment reduces modified polypeptide isoforms. Integration results are provided in Table 2.

TABLE 2
Relative Integration Percentage of Acidic Peaks

| pH 3.25 buffer | % acidic peaks | pH 3.0 buffer | % acidic peaks |
|---|---|---|---|
| Initial | 37 | Initial | 37 |
| 2 hours | 34.6 | 2 hours | 34 |
| 4 hours | 33 | 4 hours | 32.3 |
| 6 hours | 23.9 | 6 hours | 21.2 |
| 24 hours | 22 | 24 hours | 21.8 |

### EXAMPLE 14

### Large scale Acid Precipitation of Milk and Purification of D2E7

To demonstrate the operability of the present invention to treat biological samples at volumes sufficient to satisfy commercial treatment and purification demands, acid treatment of 50L of G-D2E7 containing milk was performed.

Fifty liters of milk from transgenic goats expressing D2E7, frozen at its neutral pH, was thawed in a controlled manner for <55 hours at 4° C. Acid precipitation was accomplished by adding 31 ml of 2.5M citric acid, for every liter of whole milk (1.55 L of citric acid added to 50 L of milk). The mixture was transferred into one-liter centrifuge bottles and centrifuged at 4,200 rpm, for 15 minutes at 4° C.

Centrifugation yielded a three-phase separation: a top lipid layer, a bottom casein layer and a middle liquid phase, which contained D2E7. Pushing aside the top lipid layer, the middle liquid phase was decanted from the centrifuge bottles and stored at 4° C. until all of the 1L milk aliquots had been centrifuged and separated. Due to the high casein content of milk, 51.55L of acid precipitated milk yielded 36L of centrifuged liquid phase milk (a 30% reduction in volume).

Once all of the liquid phase was separated, the solution was passed through depth filters at 4° C. to remove residual solids and to produce a clear feedstream for capture chromatography. Depth filtration consisted of three 05SP BioCap 2000 filters (Cuno Incorporated, Meriden, Conn.), placed in parallel to each other, in-line with three 60ZA BioCap 2000 filters (Cuno), which were placed in parallel to each other. One 0.2 micron Sartobran (Sartorius Corporation, Edgewood, N.Y.) filter was placed in-line, following the 60ZA filters. Prior to filtering the centrifuged milk, all filters were flushed with highly purified water, WFI, and drained. The depth-filtered solution was stored at 12° C. Product temperature was maintained below 14° C. until G-D2E7 was captured by its first chromatography step; the point of increased protein stability.

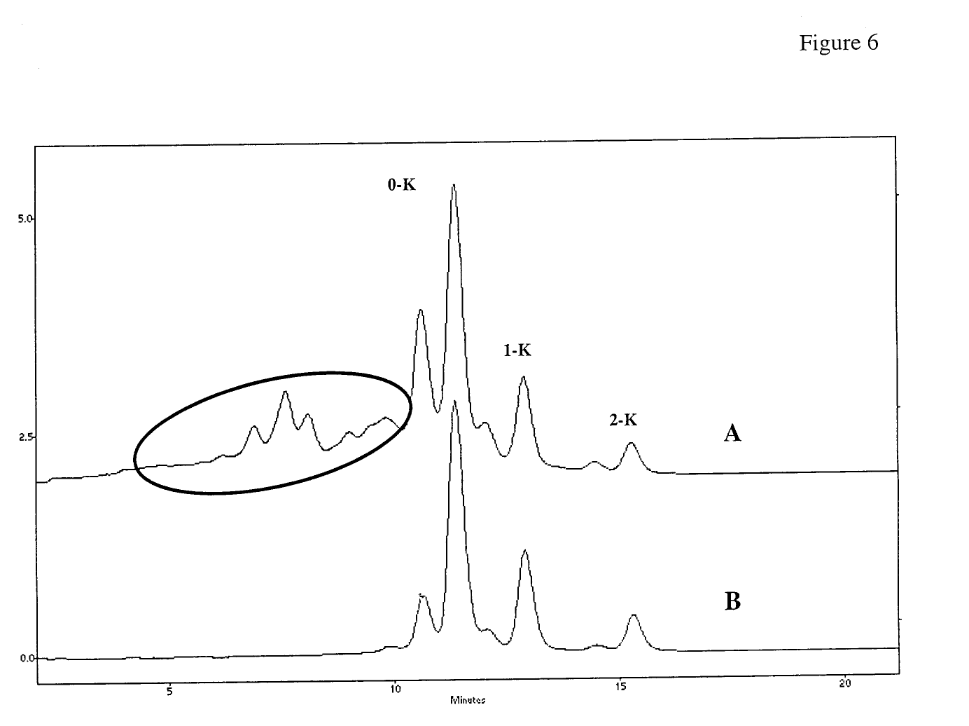

Acid precipitated G-D2E7 was extremely stable as well as fully active. FIG. 6 illustrates the stability study results of the capture step elution. Biological activity of acid precipitated G-D2E7 was assessed using the L929 bioassay performed according to the protocol described by Salfeld et al., (2000). Inhibition of cell killing by G-D2E7 in the L929 assay was 90±20% of the control. Additionally, all the G-D2E7 isoforms bound TNF-αc on the WCX-10 assay.

The acid precipitated G-D2E7 was passed over fine column chromatography including Q Sepharose FF and Phenyl Sepharose FF (Amersham Biosciences, Piscataway, N.J.). G-D2E7 processed without acidic treatment contained 42% acidic peaks (FIG. 6A). G-D2E7 processed using acidic treatment contained less than 2% acidic peaks (FIG. 6B).

These results demonstrate the operability and utility of the acid precipitation process of the present invention to reduce and to prevent polypeptide modification in solution on a large scale.

### Equivalents

Those skilled in the art will recognize, or be able to ascertain using no more than routine experimentation, many equivalents to the specific embodiments of the invention described herein. Such equivalents are intended to be encompassed by the following claims.

### REFERENCES

Bebbington and Houdebine, 1994. J. Biotechnol. 34:269-287.

Boss et al., 1984. Nucleic Acids Res. 12:3791-3806.

Brem et al., 1993. Chim. Oggi. 11:21.

Campbell et al., 1996. Nature 380: 64-66.

Catinella et al., 1996a Rapid Commun. Mass Spectrom. 10:1123.

Catinella et al., 1996b Rapid Commun. Mass Spectrom. 10:1629.

Clark et al., 1995. U.S. Pat. No. 5,476,995.

Echelard, 1996 Curr. Opin. Biotechnol. 7:536-540.

Galvani et al., 2000. Rapid Commun. Mass Spectrom. 14:1889.

Hammer et al., 1985. Nature 315:680.

Houdebine, 1995. Reprod. Nutr. Dev. 35:609-617.

Houdebine et al., 2000. Transgenic Res. 9(4-5):305-320.

J\ddot{a}nne et al., 1992. Ann. Med. 24:273.

Jones et al., 1998. J. Chromtogr. A 822:147.

Krimpenfort et al., 1991. Bio/Technology 9:88.

Krimpenfort et al., 1992. U.S. Pat. No. 5,175,384

Larsen and McEwen, (Ed.) (1998) Mass spectrometry of biological materials, 2nd edition, published by Marcel Dekker, Inc.

Meade et al., 1998. U.S. Pat. No. 5,827,690.

McBurney et al., 1964. J. Lab. Clin. Med. 64:485.

Page et al., 1996. U.S. Pat. No. 5,523,222.

Palmiter et al., 1985. Cell 41:343.

Pollock et al., 1999. J Immunological Meth. 231:147-157.

Sabbadin et al., 1999. Rapid Commun. Mass Spectrom. 13:1443.

Salfeld et al., 2000. U.S. Pat. No. 6,090,382.

Salfeld et al., 2001. U.S. Pat. No. 6,258,562 B1.

Santora et al., 1999. Anal. Biochem. 275:98-108.

Siciliano et al., 2000. Anal. Chem. 72:408.

Traldi, 1999. J. Mass Spectrom. 34:1385.

Velander et al., 1992. PNAS USA 89:12003.

Velander et al., 2002. U.S. Pat. No. 6,344,596.

Wagner et al., 1992. U.S. Pat. No. 5,175,385

Wilmut et al., 1997. Nature 385: 810-813.

Zala H. (Ed.) (1995) Expression of Antibody Genes in Mammalian Cell. In: Monoclonal Antibodies: The Second Generation. Bios scientific publishers, Oxford.

All of the publications cited herein are hereby incorporated by reference in their entirety.

We claim:

1. A method for reducing or preventing modification of a polypeptide in milk comprising steps:
a) providing milk containing a polypeptide susceptible to modification; and
b) adding acid to said milk.

2. The method according to claim 1 further comprising a step:
c) storing said milk at a temperature below room temperature.

3. The method according to claim 1 further comprising a step:
c) isolating said polypeptide from said milk.

4. The method according to claim 2 further comprising a step:
d) isolating said polypeptide from said milk.

5. The method according to claim 2 wherein said temperature is about 4° C. to about −80° C.

6. The method according to claim 5 wherein said temperature is about −20° C.

7. The method according to claim 2 wherein said polypeptide is an antibody.

8. The method according to claim 7 wherein said antibody is an anti-TNF antibody.

9. The method according to claim 8 wherein said anti-TNF antibody is D2E7.

10. The method according to claim 2 wherein said modification comprises addition of a radical group to said polypeptides, said radical group selected from the group consisting of glycosyl, glucuronidyl, peptidyl, phosphoryl, sulphuryl, farnesyl, acyl, and maleuryl.

11. The method according to claim 10 wherein said radical group is maleuryl.

12. The method according to claim 2 wherein said milk is obtained from a transgenic animal.

13. The method according to claim 12 wherein said transgenic animal is selected from the group consisting of cow, goat, sheep, pig, rat, and mouse.

14. The method according to claim 13 wherein said transgenic animal is a goat.

15. The method according to claim 2 wherein said acid is selected from the group consisting of acetic acid, citric acid, formic acid, and hydrochloric acid.

16. The method according to claim 15 wherein said acid is 2.5M citric acid.

17. The method according to claim 2 wherein said acid is added in an amount sufficient to obtain a pH of said milk of about pH 7.0 to about pH 1.0.

18. The method according to claim 17 wherein the pH of said milk is about pH 3.0 to about pH 3.5.

19. A method for reducing or preventing modification of D2E7 in milk obtained from a transgenic goat, comprising the steps:
a) providing transgenic goat milk containing D2E7;
b) adding 2.5 M citric acid to said milk in an amount sufficient to obtain a pH of said milk of about pH 3.0 to about pH 3.5; and
c) isolating said D2E7 from said milk.

20. A polypeptide isolated from milk treated according to any of the claims 1-19.
